(19) United States
(12) Patent Application Publication
(10) Pub. No.: US 2021/0322664 A1
(43) Pub. Date: Oct. 21, 2021

(54) METHOD OF TREATING THE BLOOD

(71) Applicant: Softwave Tissue Regeneration Technologies, Inc., Woodstock, GA (US)

(72) Inventors: John F. Warlick, Woodstock, GA (US); Nikolaus Hopfenzitz, Konstanz (DE)

(21) Appl. No.: 17/223,585

(22) Filed: Apr. 6, 2021

Related U.S. Application Data
(60) Provisional application No. 63/011,653, filed on Apr. 17, 2020.

(51) Int. Cl. A61M 1/38; A61M 1/36

(52) U.S. Cl. CPC A61M 1/38; A61M 1/3621; A61M 2205/058; A61M 2202/206; A61M 2202/0413

(57) ABSTRACT

A method of stimulating human blood external of a patient donor has the steps of activating an acoustic shock wave or pressure pulse generator to emit acoustic shock waves or pressure pulses directed to impinge the blood, subjecting the blood to the acoustic shock waves or pressure pulses to form stimulated blood cells and transfusing the stimulated blood cells into the patient donor. The patient donor is infected with a virus and the blood exhibits at least traces of the virus. The emitted acoustic shock waves or pressure pulses stimulating the stimulated blood cells fragment the virus in the blood. The fragmented virus in the blood transfused back into the patient donor triggers a defensive immune response to kill the virus. The emitted acoustic shock waves or pressure pulses are preferably of a low energy. The emitted shock waves or pressure pulses stimulate the blood cells and fragment the virus in the absence of cell damaging cavitation due to an elasticity in the blood cells and a lack of elasticity in the virus. The blood is not filtered. The blood can be oxygenated via Extracorporeal membrane oxygenation (ECMO) after being stimulated.

### TECHNICAL FIELD

The present invention relates to a treatment for blood, more particularly whole red blood cells, plasma or other blood components externally using sound waves, more particularly acoustic shock waves or pressure pulses.

### BACKGROUND OF THE INVENTION

Many people experience problems breathing. The main function of the lungs is the process of gas exchange called respiration or breathing. In respiration, oxygen from incoming air enters the blood and carbon dioxide, a waste gas from the metabolism, leaves the blood. A reduced lung function means that the ability of the lungs to exchange gases is reduced.

Diseases of the lungs inhibit the flow of oxygen into the blood stream which affects all the other body functions reducing the person's brain activity and physical stamina. Some common lung diseases include asthma, bronchitis, Chronic Obstructive Pulmonary Disease (COPD), cystic fibrosis, emphysema, Idiopathic pulmonary fibrosis (IPF), flu, lung cancer, obstructive sleep apnea, pleurisy, pneumonia, tuberculosis (TB). Other viral infections include: Coronavirus, Covid-19, Respiratory syncytial virus (RSV), Middle East Respiratory Syndrome (MERS), Severe acute respiratory syndrome (SARS).

Some of these diseases are treatable and can be cured, others simply can be controlled but not cured. Ideally, a cure for every type of lung disease would be possible.

The present inventive medical treatment brings the possibility to not only mitigate these diseases, but in many cases cure the patient in such a way that normal lung function is achieved. More particularly, a direct treatment of blood using sound waves eradicates bacteriological organisms, fungi and exposes virus to be unmasked allowing the bodies defense systems to destroy these viruses.

The present inventors have been involved in the development of acoustic sound waves or pressure pulses over the last decade in the treatment of tissue and organs. They have led the medical community in a variety of breakthrough medical treatments for a variety of conditions. Recently, they discovered that pressure pulses or acoustic sound waves could, contrary to the common belief, be applied directly to the thin delicate tissue membranes of the lungs. Heretofore, those skilled in the art felt that directing such energy to the lungs would risk tearing or rupturing the lung. Shields and other devices were developed to prevent this from occurring. In particular, in treating the heart with sound waves, the inventors went to great trouble to avoid an emission path that would impinge the lung sacs.

Recently, one of these same inventors discovered a unique way to overcome these concerns and treat the diseases of the lungs directly using acoustic waves and pressure pulses without risk of damage to the lung tissue. These novel methods are also described herein.

In conducting this research on lungs, directly or even indirectly, the inventors also contemplated treating the red blood cells directly, preferably externally of the patient using acoustic shock waves or pressure pulses to help purify an infected patient's blood supply.

### SUMMARY OF THE INVENTION

People who have been infected with the virus and recovered are most typically immune to that virus. This is a principle of active vaccination using non active particles of the virus. The viral envelope fragments when provided, allow the recognition of the virus to stimulate an immune reaction without having any activity of the virus.

When not fragmented, the virus can mask itself as long as it is complete. This makes it difficult for the body to develop an immune response. In the present invention, the shock waves destroy the virus into fragments because the envelope is not elastic and breaks. When this occurs, the body can recognize these fragments and develop an immune response. In addition, during the phase when the virus multiplies in the cell by the help of the cell, incomplete new viruses are in a vulnerable phase. These could also lose their capacity to mask through shock waves and will be recognised by the body and allow an immune reaction to develop.

One specific coronavirus, the COVID19-viruses protect themselves with a special envelope, which serves to protect the viruses and which is initially not recognized by the body's immune system. Through this masking, the viruses manage to gain access to the cell, where they entice the cell to reproduce the viruses. Fragments of viruses, however, can be recognised by the immune system and an adequate immune defence can be developed. Therefore, the approach of the present invention focuses on viruses in the body itself at every stage of the infection when the immune system has not yet recognized the viruses and has been unable to develop a defence to fight and unmask them. The envelope of viruses is less elastic than the membrane of cells. Shock waves have the property of exerting forces on the virus via mechanotransduction at very high speed, but the envelope of the virus does not have sufficient elasticity and breaks, the present invention uses the fragility of the fragmented envelope to eradicate the virus.

In a prior art document, U.S. Pat. No. 8,343,420 B2 entitled “Methods and Devices for Cleaning and Sterilization with Shock Waves”, this patent teaches using acoustic pressure pulses or shock waves to treat blood in containment ex vivo by employing multiple and sequential applicators to damage pathogens using significant cavitation. The high velocity cavitation microjets generated during the collapse of the cavitation bubbles play an important role in permanently breaching the membranes of bacteria and viruses and thus destroying them. The present invention achieves this in a way far less damaging to the red blood cells.

With COVID19 it is observed that the patient's immune system overreacts and attacks the organs of the patient himself. Primary unspecific immune response. This overreaction is due to the fact that the body could not recognize the viruses. By using the invention, a sufficient number of viruses are detected and a specific immune reaction is developed so that the unspecific overreaction can be avoided.

It is known from research on biofilm that shock waves are very well able to destroy the biofilm of bacteria. This breaks down the resistance of the bacteria, which is particularly high in the compound as in the biofilm.

In the course of observations of treatments of COVID19 patients, shock waves were initially applied only to the extremities in order to understand the reaction of the patients. The treatment of the extremities is based on different hypotheses. Besides the description of reflex zones hand and foot, which are activated by the treatment, anatomically also blood vessels and nerve endings are significant for these extremities. The resulting slowed capillary flow is used as an approach to treat and fragment systemic viruses. The reflexion of the waves at the numerous bones and vessels are helpful in this destructive application of viruses.

After the application of feet and hands of corona patients showed no adverse side effects but exhibited advantages. This led to the gradual increase of impulse numbers directed at the lung tissue to be performed. The combination of both these approaches has shown the best in vivo results so far.

The application of the shock waves can be done in different ways. On the one hand, the direct application to the lung tissue where a high number of viruses settle. Since viruses also occur systemically throughout the body, the application can also be applied to extremities, such as the soles of the feet and hands where the blood vessels are located a short distance below the skin surface, or to extremities with a good blood supply. Due to the low energy flow density, the application can also be done via large blood vessels either in vivo or in vitro.

The in vitro type of application is treating by sound waves directly to the blood which is conducted through external vessels. By having a certain bypass of blood flow from a body vein which is led back into the body via a large-volume “artificial” vessel. The shock wave applicator is coupled to this vessel and the shock waves are applied to the blood via a pressure permeable membrane. After 40 years of use in kidney stone fragmentation at much higher energies, there is no evidence that the blood body could be damaged by this. The white and red blood cells are elastic enough to allow the shock wave to pass without damage.

A special feature of the invention is in connection with a ventilator through which the blood of the patient is passed. This device can be supplemented by a flow-through container through which a shock wave application to the blood can take place preferably before the process of oxygen enrichment takes place.

A method of stimulating human blood external of a patient donor has the steps of activating an acoustic shock wave or pressure pulse generator to emit acoustic shock waves or pressure pulses directed to impinge the blood, subjecting the blood to the acoustic shock waves or pressure pulses to form stimulated blood cells and transfusing the stimulated blood cells into the patient donor. The patient donor is infected with a virus and the blood exhibits at least traces of the virus. The emitted acoustic shock waves or pressure pulses stimulating the stimulated blood cells fragment the virus in the blood. The fragmented virus in the blood transfused back into the patient donor triggers a defensive immune response to kill the virus. The emitted acoustic shock waves or pressure pulses are preferably of a low energy. The emitted shock waves or pressure pulses stimulate the blood cells and fragment the virus in the absence of cell damaging cavitation due to an elasticity in the blood cells and a lack of elasticity in the virus. The blood is not filtered. The blood can be oxygenated via Extracorporeal membrane oxygenation (ECMO) after being stimulated.

A composition of transfusable blood has a quantity of blood for transfusions and a plurality of fragmented viruses fragmented by acoustic shock waves or pressure pulses dispersed in the quantity of blood. The plurality of fragmented viruses when transfused into a virus infected patient activates a defensive immune response to kill the virus in the transfused patient.

The acoustic shockwave or acoustic wave generator or source can be a spherical, ballistic, radial, piezoelectric, electrohydraulic, electromagnetic or other similar device.

### Definitions

A “curved emitter” is an emitter having a curved reflecting (or focusing) or emitting surface and includes, but is not limited to, emitters having ellipsoidal, parabolic, quasi parabolic (general paraboloid) or spherical reflector/reflecting or emitting elements. Curved emitters having a curved reflecting or focusing element generally produce waves having focused wave fronts, while curved emitters having a curved emitting surfaces generally produce wave having divergent wave fronts.

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different ways, for example: A focused wave will become divergent once it has passed through the focal point. Spherical waves are also included in this definition of divergent waves and have wave fronts with divergent characteristics.

Extracorporeal membrane oxygenation (ECMO) is a technique of life support that consists of diverting a fraction of the patient's blood flow (BF) through an artificial lung for gas exchange (oxygenation and carbon dioxide [CO2] removal) and then returning it to the patient.

“Extracorporeal” occurring or based outside the living body.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plain waves. “Nearly plane” waves can be emitted by generators having pressure pulse/shock wave generating elements with flat emitters or curved emitters. Curved emitters may comprise a generalized paraboloid that allows waves having nearly plane characteristics to be emitted.

A “pressure pulse” according to the present invention is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nano-seconds (ns) up to some milli-seconds (ms). Very fast pressure pulses are called shock waves. Shock waves used in medical applications do have amplitudes above 0.1 MPa and rise times of the amplitude can be below 1000 ns, preferably at or below 100 ns. The duration of a shock wave is typically below 1-3 micro-seconds (μs) for the positive part of a cycle and typically above some micro-seconds for the negative part of a cycle. These typical time durations can be compressed by employing very high frequency devices of 1000 Hz or more while still maintaining a symmetric profile of a shock wave all of which are included within the scope of the present invention. In addition to the more common sources of shock wave or pressure pulse generators such as radial, spherical, electrohydraulic, piezoelectric and ballistic generators, the present invention contemplates laser generators. Laser generators produce numerous tiny acoustic waves as the laser beam pulses. The lower energy shock waves generated by lasers mimic the more conventional sources of sound waves and are therefore to be included herein.

“Shock Wave”: As used herein is defined by Camilo Perez, Hong Chen, and Thomas J. Matula; Center for Industrial and Medical Ultrasound, Applied Physics Laboratory, University of Washington, 1013 NE 40th Street, Seattle, Wash. 98105; Maria Karzova and Vera A. Khokhlovab; Department of Acoustics, Faculty of Physics, Moscow State University, Moscow 119991, Russia; (Received 9 Oct. 2012; revised 16 Apr. 2013; accepted 1 May 2013) in their publication, “Acoustic field characterization of the Duolith: Measurements and modeling of a clinical shock wave therapy device”; incorporated by reference herein in its entirety.

Symptomatic patient: a patient with symptoms pertaining to a symptom or symptoms, of the nature of or constituting a symptom; indicative of a disease/virus.

Asymptomatic: The term asymptomatic means literally the absence of symptoms. It describes a condition that is present, but in which a person does not show any outward signs or symptoms of the disease.

Waves/wave fronts described as being “focused” or “having focusing characteristics” means in the context of the present invention that the respective waves or wave fronts are traveling and increase their amplitude in direction of the focal point. Per definition the energy of the wave will be at a maximum in the focal point or, if there is a focal shift in this point, the energy is at a maximum near the geometrical focal point. Both the maximum energy and the maximal pressure amplitude may be used to define the focal point.

Diseases of the lung and pulmonary disorders: these can have various causes such as diseases, viruses, microorganisms, bacteria, infections, etc.

Asthma is a chronic (life time) disease that makes your lungs very sensitive and hard to breathe. Asthma can't be cured, but with proper treatment, people with asthma can lead normal, active lives. If you have asthma, your airways (breathing passages) are very sensitive. Certain things can make your airways become: Swollen and filled with mucus—the swelling and mucus makes your airways narrower, so it is hard for air to pass through; Small and tight—your airways might also become twitchy and squeeze together and tighten. This makes your airways narrower and hard for air to pass through.

Bronchitis means swelling in your air passages (bronchi). Bronchi are the air passages that connect your windpipe (trachea) with tiny air sacs (alveoli) in your lungs. The air sacs are where your body absorbs the oxygen you breathe in. Bronchitis is an inflammation of the bronchi. This inflammation means the walls of your bronchi are swollen and filled with extra sticky mucus. Airflow into and out of your lungs is partly blocked because of the swelling and extra mucus in your bronchi. This makes you cough. There are two kinds of bronchitis: Acute bronchitis makes you sick for a while, but gets better after two to three weeks. Chronic bronchitis doesn't go away. With chronic bronchitis, you have a cough with mucus most days for three months of the year.

COPD means Chronic Obstructive Pulmonary Disease. It is a term that covers two types of chronic (long-term) diseases where the airways (breathing tubes) in the lungs become swollen and partly blocked. COPD gets worse over time. It cannot be cured, but it can be treated and managed. COPD consists of two major breathing diseases: emphysema and chronic bronchitis. Emphysema damages the tiny alveoli (air sacs) at the tips of your lungs. Normally these air sacs stretch like balloons as you breathe in and out. Emphysema makes these air sacs stiff. Because they cannot stretch, air gets trapped inside them. This makes it difficult for you to breathe in and makes you feel tired. Chronic bronchitis makes your airways red, swollen and irritated. Glands in your airways make extra mucus (phlegm), which blocks some air from passing through. This makes you cough, cough up mucus and feel short of breath. Many people with COPD have both of these diseases.

Coronavirus: A coronavirus is a type of common virus that can infect your nose, sinuses, or upper throat. They can spread much like cold viruses. Almost everyone gets a coronavirus infection at least once in their life, most likely as a young child. Most coronaviruses are not dangerous, but some are. Those that cause Middle East respiratory syndrome (MERS) or severe acute respiratory syndrome (SARS) can be deadly. Influenza (the flu) and COVID-19, the illness caused by the new coronavirus, are both infectious respiratory illnesses. Although the symptoms of COVID-19 and the flu can look similar, the two illnesses are caused by different viruses.

Cystic fibrosis (CF) Cystic fibrosis mainly affects people's lungs and digestion. People with cystic fibrosis have an unusually thick, sticky mucus that clogs their lungs, makes it hard to breathe, and can lead to life-threatening lung infections. CF also affects the pancreas: thick secretions there stop the release of the digestive enzymes that normally help break down food, making it hard for people to digest and absorb nutrients. The mucus can also block the bile duct in the liver, which eventually causes permanent liver damage in some people with CF.

Emphysema is a serious respiratory disease, which is another form of COPD. The most common cause is smoking. Those who suffer from emphysema have trouble exhaling air from their lungs. Cigarette smoke damages the air sacs in the lungs to a point where they can no longer repair themselves.

Idiopathic pulmonary fibrosis (IPF) is a type of lung disease that results in scarring (fibrosis) of the lungs for an unknown reason. Over time, the scarring gets worse and it becomes hard to take in a deep breath and the lungs cannot take in enough oxygen. IPF is a form of interstitial lung disease, primarily involving the interstitium (the tissue and space around the air sacs of the lungs), and not directly affecting the airways or blood vessels. There are many other kinds of interstitial lung disease that can also cause inflammation and/or fibrosis, and these are treated differently. It is important to work with your doctor to determine if you have IPF or another form of interstitial lung disease.

The flu is a highly contagious illness caused by the influenza virus. The influenza virus causes infections of the nose, throat and lungs. In most people, the flu is uncomfortable and tiring. It can keep people in bed for days or even a couple of weeks. Some people are more at risk for serious complications from the flu, including seniors, young children, and people with long-term lung diseases like asthma and chronic obstructive pulmonary disease (COPD). Flu can make asthma symptoms worse and cause COPD flare-ups. Like the regular flu, H1N1 (swine flu) can lead to more serious problems including pneumonia, a lung infection, and other breathing problems.

Hantaviruses are a family of viruses spread mainly by rodents and can cause varied disease syndromes in people worldwide. Infection with any hantavirus can produce hantavirus disease in people. Hantaviruses in the Americas are known as “New World” hantaviruses and may cause hantavirus pulmonary syndrome (HPS).

Lung cancer is cancer that starts in the lungs. Cancer is a disease where cancer cells grow out of control, taking over normal cells and organs in the body. There are two major types of lung cancer; non-small cell cancer and small cell lung cancer. Each type of lung cancer grows and spreads in different ways. Each type may be treated differently.

Pleurisy is an inflammation of the pleura. The pleura is a two layered membrane that both encloses the lung and lines the chest cavity. People have two pleurae, one around each lung. The pleurae act as a protective wrapping, fitting snugly over your lungs. Pleurae are made up of two layers. Normally, there is no space between the inner and outer layer. The layers are joined at the edges, so that the pleura might be compared to a balloon with no air, completely empty of air and wrapped tightly around the outside of each of the lungs. Normally, there is nothing but a thin layer of lubricating layer of fluid between the inner pleural lining and the outer pleural lining. The smooth pleura linings and lubricating fluid allow your lungs to move freely in your chest, as they do in normal breathing. In people with pleurisy, the two layers of pleura get inflamed (red and swollen). This can create a space between the layers called the pleural cavity (cavity means space). In wet pleurisy, this space can fill up with fluid that can get infected.

Pneumonia (nu-MO-ne-ah) is swelling (inflammation) of one or both lungs that is usually caused by an infection. Many different germs can cause pneumonia, including bacteria, viruses, and fungi. When you breathe in these germs, they can settle in the air sacs (alveoli) of your lungs. Deep in your lungs, the germs may grow and overcome your body's normal defenses. After the lungs become infected, the air sacs (alveoli) in the lungs fill with pus and mucus. This swelling (inflammation) of the air sacs makes them less stretchy and keeps oxygen from properly reaching your blood stream.

Obstructive sleep apnea (also called OSA or obstructive sleep apnea-hypopnea syndrome) means you have short pauses in your breathing when you sleep. These breathing pauses—called apneas or apnea events—last for 10 to 30 seconds, maybe longer. People with obstructive sleep apnea can stop breathing dozens or hundreds of times each night leading to sleep disruption and low levels of oxygen.

Tuberculosis (TB) is a serious disease caused by breathing in a bacteria called Mycobacterium tuberculosis. TB usually infects the lungs. TB can also infect other parts of the body, including the kidneys, spine and brain.

### BRIEF DESCRIPTION OF THE DRAWINGS

The invention will be described by way of example and with reference to the accompanying drawings in which:

FIG. 1 is a simplified depiction of a pressure pulse/shock wave (PP/SW) generator with focusing wave characteristics.

FIG. 2 is a simplified depiction of a pressure pulse/shock wave generator with plane wave characteristics.

FIG. 3 is a simplified depiction of a pressure pulse/shock wave generator with divergent wave characteristics.

FIG. 4 is a simplified depiction of a pressure pulse/shock wave generator being connected to a control/power supply unit.

FIG. 5 shows an exemplary pressure pulse/shock wave generator device.

FIG. 5A shows an exemplary pressure pulse/shock wave generator device contacting a bag or container or tube of blood.

FIG. 6 shows a blood treatment device by way of example a dialysis device that can be used with a pressure pulse/shock wave generator device.

FIG. 7 shows an Extracorporeal membrane oxygenation (ECMO) device that can be used with a pressure pulse/shock wave generator device.

### DETAILED DESCRIPTION OF THE INVENTION

The goal in such treatments is to provide 100 to 3000 acoustic shock waves or pressure pulses at a voltage of 5 kV to 28 kV across a spark gap generator, electromagnetic, piezoelectric or a ballistic wave generator in a single treatment preferably or one or more adjuvant treatments by impinging the emitted waves on the blood.

The unfocused shock waves or pressure pulses can be of a divergent wave pattern or near planar pattern preferably of a low peak pressure amplitude and density. Typically, the energy density values of the shock waves range as low as 0.000001 mJ/mm² and having a high-end energy density of below 1.0 mJ/mm², preferably 0.40 mJ/mm² or less, more preferably 0.20 mJ/mm2 or less. The peak pressure amplitude of the positive part of the cycle should be in the rage of nano-second up to some milliseconds and its duration is below 1-3 microseconds.

The pressure pulse is much slower, a “pressure pulse” according to the present invention is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nano-seconds (ns) up to some milli-seconds (ms).

These shock wave energy transmissions are effective in stimulating a cellular response and can be accomplished without creating the cavitation bubbles in the blood. The underlying principle of these shock wave therapy methods is to stimulate the body's own natural healing capability by transfusing stimulated blood cells into an ill or infected patient.

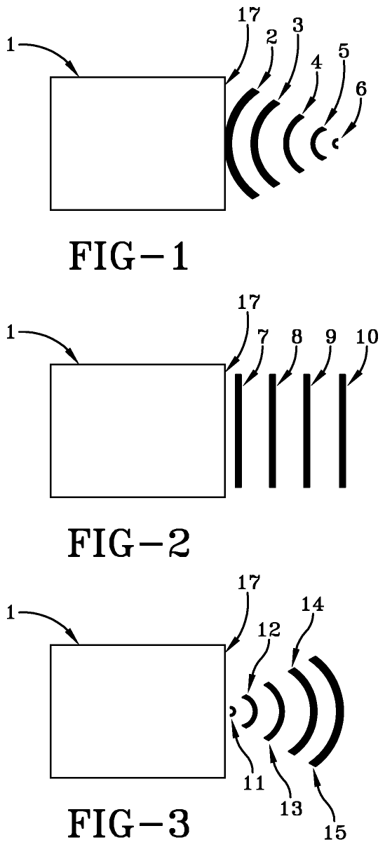

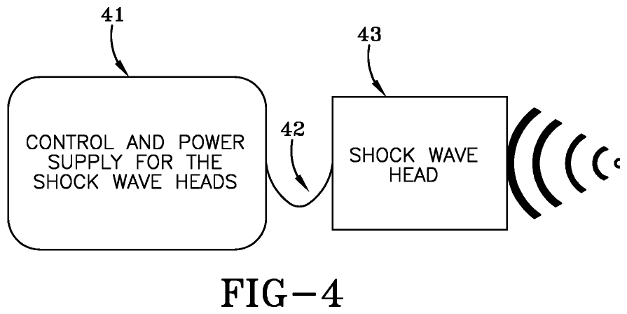

The following invention description first provides a detailed explanation of acoustic shock waves or pressure pulses, as illustrated in FIGS. 1-4. As used herein an acoustic shock wave is an asymmetric wave with an exceptionally rapid peak rise time and slower return time from the peak amplitude. Historically, these acoustic shock waves or pressure pulses were first used medically to destroy kidney stones. The wave patterns were directed to a focal point with a relatively high energy to blast the concrements into small urinary tract passable fragments.

A whole class of acoustic shock waves or pressure pulses for medical treatments were later discovered that employed low energy acoustic shock waves or pressure pulses. These low energy acoustic shock waves or pressure pulses maintained the asymmetric wave profile, but at much lower energies as described in US2006/0100550 which is incorporated herein in its entirety.

These low energy acoustic shock waves or pressure pulses advantageously could stimulate blood without requiring a focused beam. The advantage of such an unfocused beam was the acoustic wave could be directed to pass through a container or tubing filled with blood without causing any cell rupturing which would be evidenced by a lack of cell membrane damage. This use of unfocused, low energy acoustic shock waves or pressure pulses provided an ability to treat a large volume of blood.

The use of low energy acoustic shock waves or pressure pulses that employ a focused beam has been spurred on as a viable alternative to the unfocused low energy shock waves because the focal point being of a small point of energy has little or a small region of cell damage as the remaining portions of the wave pattern can provide a stimulating effect similar to the unfocused shock waves. Basically, the effect is the same with the users of focused waves achieving the benefits of the unfocused waves, but with a focal point of peak energy in a tiny localised region. So, for purposes of the present invention, the use of “soft waves” those defined by low energy beams will be applicable to both focused and unfocused beams of acoustic shock waves or pressure pulses for the present invention.

One last and significant point that the reader must appreciate is that an “acoustic shock wave” is not an “ultrasound wave”. Sonic or ultrasound waves are generated with a uniform and symmetrical wave pattern similar to a sinusoidal wave. This type of sonic wave causes a sheer action on tissue as evidenced by a generation of heat within the tissue or blood, for this reason, the use of sonic waves of the ultrasonic type are not considered as efficient in cell survivability rates. The present invention provides an apparatus for an effective treatment of indications, which benefit from high or low energy pressure pulse/shock waves having focused or unfocused, nearly plane, convergent or even divergent characteristics. With an unfocused wave having nearly plane, plane, convergent wave characteristic or even divergent wave characteristics, the energy density of the wave may be or may be adjusted to be so low that side effects including pain are very minor or even do not exist at all.

In certain embodiments, the apparatus of the present invention is able to produce waves having energy density values that are below 0.1 mJ/mm² or even as low as 0.000 001 mJ/mm². In a preferred embodiment, those low-end values range between 0.1-0.001 mJ/mm². With these low energy densities, side effects are reduced, and the dose application is much more uniform. The apparatus of the present invention also may allow the user to make more precise energy density adjustments than an apparatus generating only focused shock waves, which is generally limited in terms of lowering the energy output.

The present invention relates to the use of various therapeutic pressure pulse wave patterns or acoustic shock wave patterns as illustrated in FIGS. 1-3 for treating the blood of infected donors and using the treated blood for direct transfusion back into the infected donor or if properly blood type matched into another patient. Each illustrated wave pattern will be discussed later in the description; however, the use of each has particularly interesting beneficial features that are a remarkably valuable new tool in the fight against such viruses.

With reference to FIGS. 1-3, a variety of schematic views of acoustic shock waves or pressure pulses are described. The following description of the proper amplitude and pressure pulse intensities of the shock waves are provided along with a description of how the shock waves actually function. For the purpose of describing, the shock waves were used as exemplary and are intended to include all of the wave patterns discussed in the figures as possible treatment patterns.

FIG. 1 is a simplified depiction of a pressure pulse/shock wave (PP/SW) generator, such as a shock wave head, showing focusing characteristics of transmitted acoustic pressure pulses. Numeral 1 indicates the position of a generalized pressure pulse generator, which generates the pressure pulse and, via a focusing element, focuses it outside the housing to treat diseases. The affected blood is generally located in or near the focal point which is located in or near position 6. At position 17 a water cushion or any other kind of exit window for the acoustical energy is located.

FIG. 2 is a simplified depiction of a pressure pulse/shock wave generator, such as a shock wave head, with plane wave characteristics. Numeral 1 indicates the position of a pressure pulse generator according to the present invention, which generates a pressure pulse which is leaving the housing at the position 17, which may be a water cushion or any other kind of exit window. Somewhat even (also referred to herein as “disturbed”) wave characteristics can be generated, in case a paraboloid is used as a reflecting element, with a point source (e.g. electrode) that is located in the focal point of the paraboloid. The waves will be transmitted into a bag or container of blood via a coupling media such as, e.g., ultrasound gel or oil and their amplitudes will be attenuated with increasing distance from the exit window 17.

FIG. 3 is a simplified depiction of a pressure pulse shock wave generator (shock wave head) with divergent wave characteristics. The divergent wave fronts may be leaving the exit window 17 at point 11 where the amplitude of the wave front is very high. This point 17 could be regarded as the source point for the pressure pulses. In FIG. 3 the pressure pulse source may be a point source, that is, the pressure pulse may be generated by an electrical discharge of an electrode under water between electrode tips. However, the pressure pulse may also be generated, for example, by an explosion, referred to as a ballistic pressure pulse. The divergent characteristics of the wave front may be a consequence of the mechanical setup.

This apparatus, in certain embodiments, may be adjusted/modified/or the complete shock wave head or part of it may be exchanged so that the desired and/or optimal acoustic profile such as one having wave fronts with focused, planar, nearly plane, convergent or divergent characteristics can be chosen.

A change of the wave front characteristics may, for example, be achieved by changing the distance of the exit acoustic window relative to the reflector, by changing the reflector geometry, by introducing certain lenses or by removing elements such as lenses that modify the waves produced by a pressure pulse/shock wave generating element. Exemplary pressure pulse/shock wave sources that can, for example, be exchanged for each other to allow an apparatus to generate waves having different wave front characteristics are described in detail below.

In one embodiment, mechanical elements that are exchanged to achieve a change in wave front characteristics include the primary pressure pulse generating element, the focusing element, the reflecting element, the housing and the membrane. In another embodiment, the mechanical elements further include a closed fluid volume within the housing in which the pressure pulse is formed and transmitted through the exit window.

In one embodiment, the apparatus of the present invention is used in combinations of shock wave therapies. Here, the characteristics of waves emitted by the apparatus are switched from, for example, focused to divergent or from divergent with lower energy density to divergent with higher energy density. Thus, effects of a pressure pulse treatment can be optimized by using waves having different characteristics and/or energy densities, respectively.

While the above described universal toolbox of the various types of acoustic shock waves or pressure pulses and types of shock wave generating heads provides versatility, the person skilled in the art will appreciate that apparatuses that produce low energy or soft acoustic shock waves or pressure pulses having, for one example, nearly plane characteristics, are less mechanically demanding and fulfill the requirements of many users.

As the person skilled in the art will also appreciate that embodiments shown in the drawings are independent of the generation principle and thus are valid for not only electro-hydraulic shock wave generation but also for, but not limited to, PP/SW generation based on electromagnetic, piezoceramic and ballistic principles. The pressure pulse generators may, in certain embodiments, be equipped with a water cushion that houses water which defines the path of pressure pulse waves that is, through which those waves are transmitted. In a preferred embodiment, a container or tubing filled with blood is coupled via ultrasound gel or oil to the acoustic exit window (17), which can, for example, be an acoustic transparent membrane, a water cushion, a plastic plate or a metal plate.

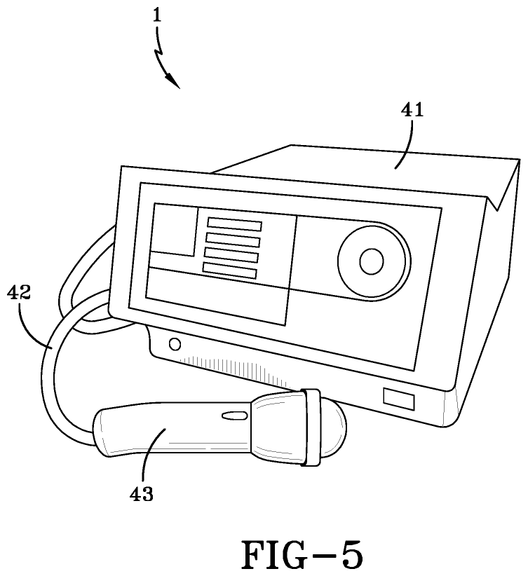

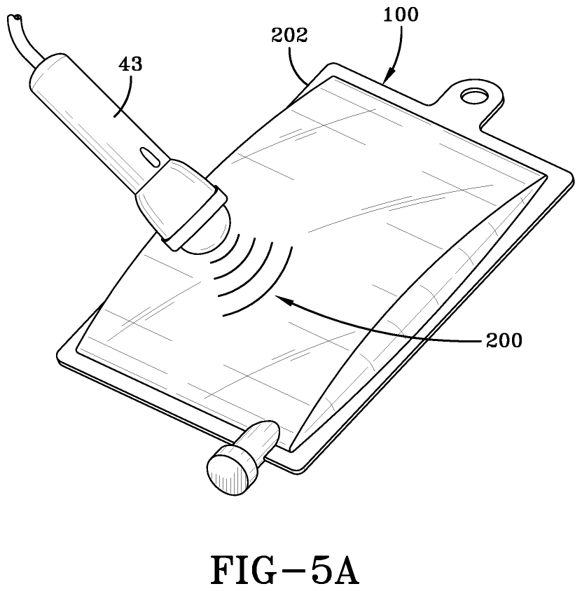

FIG. 5 shows an exemplary shock wave device generator or source 1 with a control and power supply 41 connected to a hand-held applicator shock wave head 43 via a flexible hose 42 with fluid conduits. The illustrated shock wave applicator 43 has a flexible membrane at an end of the applicator 43 which transmits the acoustic waves when coupled to the skin by using a fluid or acoustic gel. As shown, this type of applicator 43 has a hydraulic spark generator using either focused or unfocused shock waves, preferably in a low energy level, less than the range of 0.01 mJ/mm² to 0.3 mJ/mm². The flexible hose 42 is connected to a fluid supply that fills the applicator 43 and expands the flexible membrane when filled. Alternatively, a ballistic, piezoelectric or spherical acoustic shock wave device can be used to generate the desired waves.

FIG. 5A shows the applicator 43 emitting waves 200 into a container 202 of blood 100.

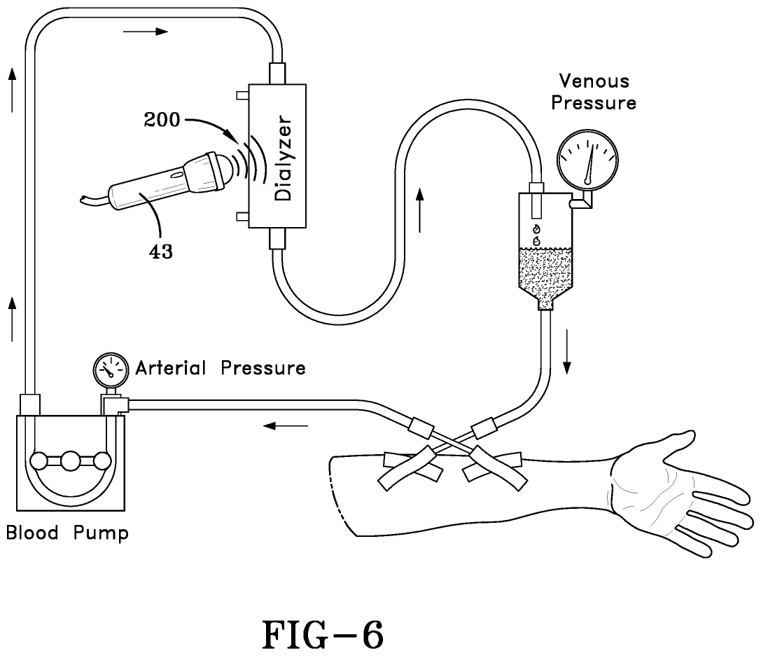

FIG. 6 shows a blood treatment device by way of example a dialysis device that can be used with a pressure pulse/shock wave generator device treating the blood as it passes through the tubing.

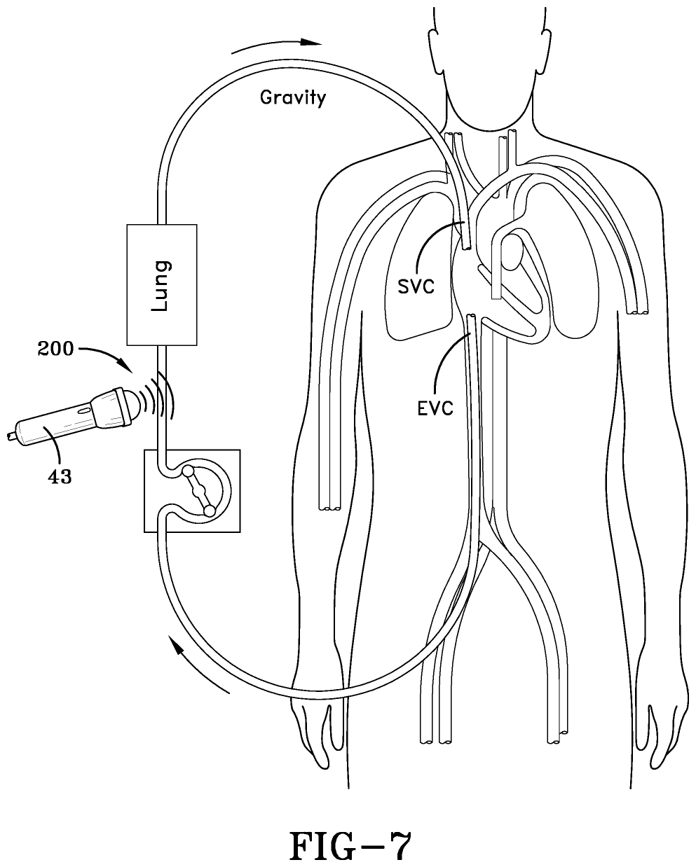

With reference to FIG. 7, a perspective view of an ECMO device is shown that can be used with a pressure pulse or shock wave generator device.

A shock wave applicator head 43 is brought into contact with the container or tube of blood 100 preferably an acoustic gel is used to enhance the transmission of the shock waves 200 through the blood 100. The shock wave applicator head 43 is connected via cabling 42 to a power generating unit 41 as shown. The shock wave applicator head 43 can be attached rigidly to a fixture or stand or alternatively can be hand held and manipulated across the blood to drive the shock waves 200 in the direction the shock wave head 43 is pointed to activate a response.

Shock waves are a completely different technology and a quantum leap beyond other forms of treatments. The mechanism of shock waves is far from being understood, but is known to cause new blood vessels to grow in an area of treatment and regenerate bony tissue. In the present invention shock waves are used to treat patients with a virus or blood disease. This is a phenomenal advancement in the current approach.

The present invention employs the use of pressure pulses or shock waves to stimulate blood.

The transmission dosage can be from a few seconds to 20 minutes or more dependent on the condition. Preferably the waves are generated from an unfocused or focused source. The unfocused waves can be divergent, planar or near planar and having a low pressure amplitude and density in the range of 0.00001 mJ/mm² to 1.0 mJ/mm² or less, most typically below 0.2 mJ/mm². The focused source preferably can use a diffusing lens or have a far-sight focus to minimize if not eliminate having the localized focus point within the tissue. Preferably the focused shock waves are used at a similarly effective low energy transmission or alternatively can be at higher energy but wherein the target site is disposed pre-convergence inward of the geometric focal point of the emitted wave transmission. This emitted energy preferably stimulates the blood cells with minimal rupturing of cellular membranes. The surrounding healthy blood cells in the region treated are activated and the virus is fragmented so upon transfusion an immune defence mechanism response is initiated.

As illustrated, the device shown is an electrohydraulic acoustic shock wave generator, however, other devices that generate acoustic shock waves or pressure pulses can be used. Ultrasonic devices may be considered, but there is no data to support a sinusoidal wave form would work and therefore not considered as effective as the asymmetric wave generators. The acoustic shock waves or pressure pulses activate a cellular response within the blood. This response or stimulation causes an increase of nitric oxide and a release of a variety of growth factors such as VEGF and a release of anti-microbial peptides like LL37. As shown, the flexible membrane is protruding outward and the applicator 43 has been filled with fluid, the transmission or emission of acoustic shock waves or pressure pulses 200 is directed towards the blood 100. It is believed that a single treatment of the blood 100 will achieve the desired effect. However, repeated treatments may be administered.

These shock wave energy transmissions are effective in stimulating a cellular response and can be accomplished without creating excessive cavitation bubbles in the blood when employed in other than site targeted high energy focused transmissions. This effectively ensures the blood cells do not have to experience the sensation of tearing or of excessive hemorrhaging so common in the use of higher energy focused wave forms having a focal point at or within the targeted treatment site.

Due to the wide range of beneficial treatments available it is believed preferable that the optimal use of one or more wave generators or sources should be selected on the basis of the specific application. Wherein relatively small target sites may involve a single wave generator placed on an adjustable manipulator arm. A key advantage of the present inventive methodology is that it is complimentary to conventional medical procedures.

The underlying principle of these pressure pulse or shock wave therapy methods is to enrich the blood directly while fragmenting the virus creating an effective vaccine effect. This is particularly useful in containing asymptomatic patients who carry the virus and to stimulate the body's own natural healing capability. The acoustic shock waves transmit or trigger what appears to be a cellular communication throughout the entire anatomical structure, this activates a generalized cellular response at the blood, in particular, but more interestingly a systemic response in areas more removed from the wave form pattern. This is believed to be one of the reasons molecular stimulation can be conducted at threshold energies heretofore believed to be well below those commonly accepted as required. Accordingly, not only can the energy intensity be reduced in some cases, but also the number of applied shock wave impulses can be lowered from several thousand to as few as one or more pulses and still yield a beneficial stimulating response. The key is to provide at least a sufficient amount of energy to activate healing reactions and fragment the virus in the blood.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A method of stimulating human blood external of a patient donor comprises the steps of:
activating an acoustic shock wave or pressure pulse generator to emit acoustic shock waves or pressure pulses directed to impinge the blood;
subjecting the blood to the acoustic shock waves or pressure pulses to form stimulated blood cells; and
transfusing the stimulated blood cells into the patient donor.

2. The method of claim 1 wherein the patient donor is infected with a virus and the blood exhibits at least traces of the virus.

3. The method of claim 2 wherein the emitted acoustic shock waves or pressure pulses stimulating the stimulated blood cells fragment the virus in the blood.

4. The method of claim 3 wherein the fragmented virus in the blood transfused back into the patient donor triggers a defensive immune response to kill the virus.

5. The method of claim 1 wherein the emitted acoustic shock waves or pressure pulses are of a low energy.

6. The method of claim 1 wherein the emitted shock waves or pressure pulses stimulate the blood cells and fragment the virus in the absence of cell damaging cavitation due to an elasticity in the blood cells and a lack of elasticity in the virus.

7. The method of claim 1 wherein the blood is not filtered.

8. The method of claim 1 wherein the blood can be oxygenated via Extracorporeal membrane oxygenation (ECMO) after being stimulated.

9. A composition of transfusable blood comprises:
a quantity of blood for transfusions; and
a plurality of fragmented viruses fragmented by acoustic shock waves or pressure pulses dispersed in the quantity of blood.

10. The composition of claim 9 wherein the plurality of fragmented viruses when transfused into a virus infected patient activates a defensive immune response to kill the virus in the transfused patient.
